(19) United States
(12) Patent Application Publication
(10) Pub. No.: US 2004/0116973 A1
(43) Pub. Date: Jun. 17, 2004

(54) Heart stimulating device that initiates variably-timed threshold searches

(75) Inventor: Magnus Ohman, Hasselby (SE)

Correspondence Address: SCHIFF HARDIN, LLP, PATENT DEPARTMENT, 6600 SEARS TOWER, CHICAGO, IL, 60606-6473, US

(73) Assignee: St. Jude Medical AB

(21) Appl. No.: 10/692,109

(22) Filed: Oct. 23, 2003

(30) Foreign Application Priority Data
Dec. 16, 2002 (SE) 0203725-7

(51) Int. Cl. A61N 1/37

(52) U.S. Cl. 607/27

(57) ABSTRACT

A heart stimulating device has a stimulation threshold measuring circuit for performing threshold searches by measuring a stimulation threshold value, a pulse generator for delivering stimulation pulses of variable amplitudes and duration, a control unit for controlling the pulse generator to deliver stimulation pulses of amplitudes related to the measured threshold value, an interval timer for initiating time-initiated threshold searches, wherein the time between consecutive time-initiated threshold searches is defined as a threshold search interval. The interval timer varies the threshold search interval, dependent on the result of at least the last performed threshold search.

### BACKGROUND OF THE INVENTION

1. Field of the Invention

The present invention relates to a heart stimulating device of the type having a threshold measuring circuit and a timer that initiates a threshold search at timed intervals.

2. Description of the Prior Art

To reduce the energy consumption of heart stimulators, it is known to provide an automatic threshold search function, a so called AUTOCAPTURE™ pacing system function, to maintain the energy of the stimulation pulses at a level just above that which is needed to effectuate capture, cf. e.g. U.S. Pat. No. 5,458,623.

A threshold search is a safety measure to ensure that each stimulation pulse leads to capture and the reason for performing a threshold search is that the threshold value, i.e. the smallest amount of energy resulting in depolarization of the myocardium, is not constant.

A threshold value is often referred to as an amplitude value, i.e. the voltage at which the pacemaker stimulates. In fact, it is the strength and duration of current that reaches the heart muscle cells that determines whether the muscle will contract. The strength and duration is determined by the amplitude, pulse width and the characteristics of the electrode tip, as well as its position in relation to the myocardium.

In one usual implementation of a threshold search algorithm, the pulse width is programmed to a constant value at implantation or follow-up and is not altered by the threshold search function.

Herein, the threshold value is often exemplified as the voltage value but it should be remembered that it is also possible to alter the pulse width with a constant amplitude value.

Many different threshold search algorithms are known. The principles of a preferred threshold search algorithm will therefore only be briefly described. In a preferred algorithm the AV-interval is shortened in order to override any intrinsic heart activity and the ventricular stimulation amplitude is successively stepped down by a predetermined amplitude step of e.g. 0.3 V.

The threshold value is defined by the AUTOCAPTURE function as the lowest amplitude capable of initiating depolarisation twice in succession. The value can change both upwardly and downwardly.

If the threshold value increases, the pacemaker notices it immediately as there is no capture. With evoked response detection, the pacemaker is immediately informed that no capture has occurred. If it happens twice in succession, the pacemaker increases the amplitude by 0.3 volt and it now looks for an amplitude from which it can begin threshold-value detection again. To do this, it needs to identify two captures in succession. If capture occurs every other time, it continues to stimulate at the same amplitude. Every time there are two successive losses, it raises the amplitude.

If the pacemaker has not detected an evoked response after 62.5 ms after the stimulation pulse, it concludes that there was no capture. A back-up safety pulse is then immediately delivered with high energy. The back-up pulse may have an amplitude of 4.5 V and a width of 0.49 ms (compared to a normal stimulation pulse that has a pulse width in the order of 0.2-0.3 ms).

On the other hand, if the threshold value falls, the pulse generator will adjust the amplitude accordingly (however not immediately). It measures the threshold value regularly to be able to benefit from the saving of energy that a lower threshold value makes possible. This is done at eight-hour intervals.

When the threshold value is measured, the amplitude is reduced 0.3 volt at a time until it looses capture twice in succession. It then returns to the amplitude 0.3 volt above, to confirm that it really is the threshold value. This is confirmed by two successive captures.

Assume that the threshold value prior to detection was 1.2 volts. In that case, the pacemaker will have stimulated at 1.5 volts. The amplitude is first reduced to 1.2 volts. Two captures in succession confirm that it is not a value below the threshold. The pacemaker then tries an amplitude 0.3 volts lower, i.e. 0.9 volt. If there are two captures at this amplitude as well, the pacemaker continues downwards and tries 0.6 volt. If there are then two successive no captures, it means it has gone below the threshold. The amplitude is raised 0.3 volt again to 0.9 volt. If there are again two captures, it confirms that 0.9 volt is now the threshold value. The new stimulation amplitude is the threshold value plus a working margin, e.g. 0.3 volt, i.e. in this case, 1.2 volts.

Thus, the rule is to seek the lowest amplitude that gives two successive captures and then establish the new stimulation amplitude at the value plus a safety margin of e.g. 0.3 volt.

A heart stimulation is commercially available from St Jude Medical AB with a threshold search capability that uses a threshold search algorithm that operates in line with the threshold search algorithm described above and where the time between automatic testing of the stimulation threshold is fixed to 8 hours.

It is well known in the literature and through studies that the stimulation threshold changes during the first month after implantation and that for the following life longevity of the device, the stimulation threshold is constant unless there are special events.

It has been found that threshold searches with associated loss of capture and backup stimulation may have pro-arrhythmic effects resulting in patient discomfort due to initiated arrhythmias.

### SUMMARY OF THE INVENTION

An object of the present invention is to further optimize the threshold search algorithm in order to minimize the potential discomfort for the patient.

This object is achieved in accordance with the present invention by an automatic variation of the time interval between time-initiated threshold searches. This automatic threshold search interval regulation may be based on the historical data from the result of previous time-initiated threshold searches. The threshold interval regulation optionally also may be based on the result from Loss-of Capture (LOC) initiated threshold searches.

Thus, according to the present invention a variable threshold search interval is introduced.

In the present invention, any method to modify the stimulation pulse energy, to determine the threshold value, can be used.

Minimum and maximum time intervals can be fixed or programmable by the user. Typical values for minimum time can be 8 hours as is presently used in the devices today, and the maximum time 1 month.

The beat to beat threshold search function with back-up pulses ensures that there is always capture and in the event of unexpected threshold rises that the stimulation amplitude is increased to a new value that provides capture stimulations.

The need for the time-initiated threshold searches is to follow a declining threshold so that the stimulation is made with the lowest energy required. If the threshold is constant, the time between threshold searches therefore can be prolonged without negative impact.

### DESCRIPTION OF THE DRAWINGS

FIG. 1 is a simplified block diagram of a heart stimulating device according to the present invention.

FIG. 2 is a flow diagram illustrating the basic steps performed by a preferred embodiment of the present invention.

### DESCRIPTION OF THE PREFERRED EMBODIMENTS

As indicated in the background section a threshold search may be initiated for two different reasons. One is a time-initiated threshold search and the other is a loss-of capture (LOC) initiated threshold search.

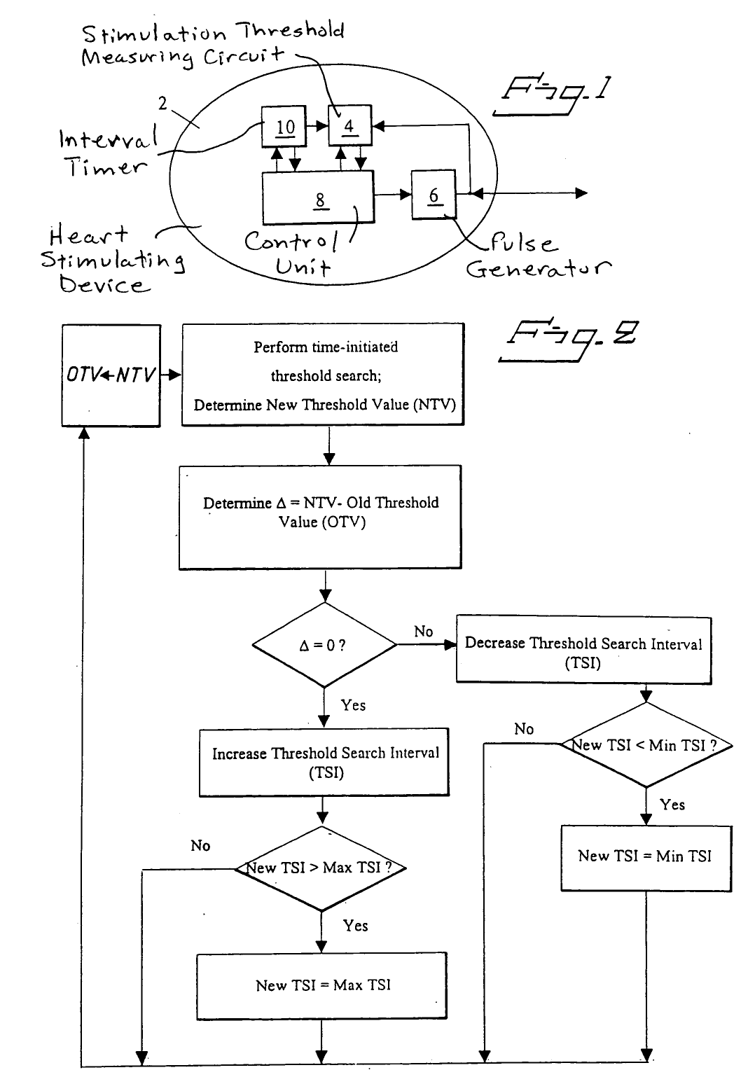

The present invention will now be described by referring to FIG. 1 that shows a simplified block diagram of a heart stimulating device according to the present invention. FIG. 1 shows a heart stimulating device 2 having a stimulation threshold measuring circuit 4 for performing threshold searches by measuring a stimulation threshold value, a pulse generator 6 for delivering stimulation pulses of variable amplitudes and duration, a control unit 8 for controlling the pulse generator to deliver stimulation pulses of amplitudes related to the measured threshold value, and an interval timer 10 for initiating time-initiated threshold searches. The time between consecutive time-initiated threshold searches is defined as a threshold search interval.

According to the present invention the interval timer 10 varies the threshold search interval, dependent on the result of at least the last performed threshold search.

The threshold value is changed in accordance with the following general rules:

If the threshold value did not change at the last performed threshold search, the threshold search interval is increased.

If the threshold value did change at the last performed threshold search, the threshold search interval is decreased.

In a preferred embodiment of the present invention the threshold search interval is variable by increasing or decreasing the interval length with a predetermined percentage of the threshold search interval. A preferred percentage is in the range 5-25% of the present threshold search interval.

As an alternative, the threshold search interval could be varied by increasing or decreasing the interval length with a preset time. The preset time is in the range 1 hour-4 days.

The maximal threshold search interval is preferably one month and the minimum threshold search interval is preferably one hour.

As indicated in the background section a threshold search is also initiated, in addition to the time initiated threshold search, if loss of capture is detected. The thresholds before and after all loss of capture initiated threshold searches between the previous and the present time initiated threshold searches are checked, and the results of these checks are used when varying the threshold search interval length. In this situation the threshold search interval is increased if the thresholds before and after all loss of capture initiated threshold searches are equal.

According to an improvement of the preferred embodiment of the present invention in the case that a threshold change has been made, the threshold search interval is maintained, for a predetermined time period after the threshold change, at a duration shorter or equal than a predetermined threshold search interval.

The predetermined threshold search interval is preferably 8 hours and the predetermined time period is preferably longer or equal one week.

FIG. 2 is a flow diagram illustrating the basic steps performed by a preferred embodiment of the present invention.

When the present Threshold Search Interval (TSI) ends, a new time-initiated threshold search is performed, by the stimulation threshold measuring means 4 (in FIG. 1), and a new Threshold Value (NTV) is determined.

The difference Δ between the NTV and the Old Threshold Value (OTV) is determined by the control means 8 and it is checked whether Δ equals 0.

If Δ equals 0, meaning that no threshold change is determined, the TSI is increased by the interval timer 10.

The New TSI is compared, in the interval timing means 10, to a maximal acceptable TSI and if the new TSI is greater than max TSI the new TSI is set to max TSI.

If Δ not equals 0, meaning that a threshold change is determined, the TSI is decreased by the interval timer 10.

The new, decreased TSI is compared, in the interval timer 10, to a minimal acceptable TSI and if the new TSI is smaller than min TSI the new TSI is set to max TSI. As soon as the above procedure has ended the new TSI starts by the interval timer 10.

Before the next threshold search is due, an Old Threshold Value (OTV) is given the value of the New Threshold Value (NTV).

FIG. 2 shows only the basic features of the present invention and many of the different embodiments discussed in relation to FIG. 1 are not shown.

Although modifications and changes may be suggested by those skilled in the art, it is the intention of the inventor to embody within the patent warranted hereon all changes and modifications as reasonably and properly come within the scope his contribution to the art.

I claim as my invention:

1. A heart stimulating device comprising:
a pulse generator which emits stimulation pulses of variable energy content;
a stimulation threshold measuring circuit adapted to interact with a subject to which the stimulation pulses are delivered, to measure a stimulation threshold value in the subject;
a timer connected to said stipulation threshold measuring circuit for initiating multiple threshold searches at respective times, said timer varying an interval between successive threshold searches dependent on a result of at least a last-performed threshold search; and
a control unit connected to said pulse generator and to said stimulation threshold measuring circuit for causing said pulse generator to emit said stimulation pulses with an energy content dependent on said stimulation threshold value measured by said stimulation threshold measuring circuit.

2. A heart stimulating device as claimed in claim 1 wherein said timer, if said stimulation threshold value did not change in said last-performed threshold search, increases said interval.

3. A heart stimulating device as claimed in claim 1 wherein said timer, if said stimulation threshold value changed in said last-performed threshold search, decreases said interval.

4. A heart stimulating device as claimed in claim 1 wherein said timer varies said interval by altering a duration of said interval with a preset timed amount.

5. A heart stimulating device as claimed in claim 1 wherein said timer varies said interval by decreasing a duration of said interval by a predetermined percentage of said interval.

6. A heart stimulating device as claimed in claim 1 wherein said timer is limited to a maximum duration for said interval of one month.

7. A heart stimulating device as claimed in claim 1 wherein said timer is limited to a minimum duration of said interval of one hour.

8. A heart stimulating device as claimed in claim 1 wherein said stipulation threshold measuring circuit also makes loss of capture-initiated threshold searches and at wherein said timer uses all loss of capture-initiated threshold searches, during said interval, to vary said interval, in addition to said threshold searches initiated by said timer.

9. A heart stimulating device as claimed in claim 8 wherein said timer increases said interval if the respective stimulation threshold values, before and after each loss of capture-initiated threshold search, are equal.

10. A heart stimulating device as claimed in claim 8 wherein said timer decreases said interval if the respective stimulation threshold values before and after any loss of capture-initiated search are not equal.

11. A heart stimulating device as claimed in claim 1 wherein said timer maintains said interval at a duration shorter than or equal to a predetermined interval, for a predetermined time period after a change of said stimulation threshold value.

12. A heart stimulating device as claimed in claim 11 wherein said timer employs an interval of eight hours as said predetermined interval.

13. A heart stimulating device as claimed in claim 11 wherein said timer employs a duration of at least one week as said predetermined time period.
